(19) United States
(12) Patent Application Publication
(10) Pub. No.: US 2006/0275356 A1
(43) Pub. Date: Dec. 7, 2006

(54) Pharmaceutical compositions for treating or preventing coronary artery disease

(76) Inventors: James Walter Burgess, Nepean (CA); Daniel L. Sparks, Gatineau (CA); Philip Alexander Sinclair, Ottawa (CA)

Correspondence Address: SMART & BIGGAR;P.O. BOX 2999, STATION D, 900-55 METCALFE STREET, OTTAWA, ON, K1P5Y6, CA

(21) Appl. No.: 11/438,334

(22) Filed: May 23, 2006

Related U.S. Application Data
(60) Provisional application No. 60/684,212, filed on May 25, 2005.

(51) Int. Cl. A61K 31/401; A61K 31/366; A61K 31/22; A61K 9/127

(52) U.S. Cl. 424/450; 514/423; 514/460; 514/548

(57) ABSTRACT

The present invention provides pharmaceutical compositions and methods for raising plasma levels of apolipoprotein A-I in a mammal, e.g. for treating or preventing coronary artery disease (CAD). The inventive pharmaceutical compositions comprise: (a) a negatively charged phospholipid; and (b) at least one intestinal absorption enhancer; where (a) and (b) are present in such composition in amounts that render the combination effective for raising plasma levels of apolipoprotein A-I in a mammal. Also provided are corresponding methods of medical treatment comprising administering to a mammal: (a) a negatively charged phospholipid; and (b) at least one intestinal absorption enhancer; where (a) and (b) are administered in amounts that render the combination effective for raising plasma levels of apolipoprotein A-I in said mammal.

### CROSS-REFERENCE TO RELATED APPLICATIONS

This application claims priority from U.S. Ser. No. 60/684,212 entitled “Pharmaceutical Compositions for Treating or Preventing Coronary Artery Disease” filed May 25, 2005.

### FIELD OF THE INVENTION

The present invention relates to pharmaceutical compositions and methods for raising plasma levels of apolipoprotein A-I (apoA-1) in a mammal. These compositions and methods find utility for example in treating or preventing coronary artery disease (CAD) in mammals.

### BACKGROUND

Coronary artery disease (CAD) is the leading cause of mortality and morbidity in the USA and most Western countries. As such, CAD is a world-wide health concern.

Most CAD is due to atherosclerosis (a condition characterized by subintimal thickening due to deposition of atheromas) of the large and medium-sized arteries of the heart.

Plasma levels of high-density lipoprotein cholesterol (HDL-C) are inversely associated with CAD (Gordon, D. J. and Rifkind, B. M. (1989) N. Engl. J. Med. vol. 321: 1311-1316). Results from controlled intervention trials suggest that a 1% increase in HDL-C corresponds to a 3% reduction in risk of developing CAD (Manninen, V. et al. (1988) JAMA vol. 260: 641-651). HDL-C is often referred to in the field as the “good” cholesterol.

HDL-C plays a key role in facilitating reverse cholesterol transport, which is a process that removes excess cholesterol from body tissues including the atheromatous plaque (Eisenberg, S. (1984) J. Lipid Res. vol. 25: 1017-1058; Fielding, C. J., et al. (1995) J. Clin. Invest vol. 95: 611-618). Therefore, higher plasma levels of HDL-C may play an anti-atherogenic role by promoting the clearance of cholesterol from the body.

The cardioprotective actions of HDL-C are commonly believed to be associated with apolipoprotein A-I (apoA-I), the major protein constituent of HDL-C (Berliner, J. A., et al. (1995) Circulation vol. 91: 2488-2496; Kawashiri, M. A., et al. (2000) Curr. Atheroscler. Rep. vol. 2: 363-372; Andersson, L. O. (1997) Curr. Opin. Lipidol. vol. 8: 225-228). ApoA-I constitutes 70% of the total protein of HDL-C, and the synthesis of apoA-I is directly linked to the levels of HDL-C in the body. Thus, high plasma apoA-I levels are tantamount to high plasma HDL-C levels, as are the cardioprotective effects stemming therefrom. The biochemical pathways involved in the regulation of apoA-I are therefore of great therapeutic interest, and there have been many attempts to raise HDL-C levels by increasing apoA-I levels.

Nicotinic acid (Niacin) remains the most widely used HDL-C raising therapy today (Kamanna, V. S. and Kashyap, M. L. (2000) Curr. Atheroscler. Rep. vol. 2: 36-4; Malik, S. and Kashyap, M. L. (2003) Curr. Cardiol. Rep. vol. 5: 470-476). Niacin decreases the catabolism of HDL-C particles through a decrease in apoA-I uptake by the liver (Jin, F. Y., Kamanna, V. S., and Kashyap, M. L. (1997) Arterioscler. Thromb. Vasc. Biol. vol. 17: 2020-2028). However, many patients experience unpleasant flushing when taking niacin. Also, niacin therapy may be counter indicated or inappropriate in patients suffering from other medical conditions.

Fibrates act as agonists of the peroxisome proliferator activated receptor (PPAR), to regulate the expression of the many genes involved in reverse cholesterol transport. Fibrates are capable of increasing the transcription of apoA-I, resulting in an increase in its hepatic synthesis (Watts, G. F., et al. (2003) Diabetes vol. 52: 803-811; Fruchart, J. C. (2001) Am. J. Cardiol. vol. 88: 24N-29N). However, fibrates do have some unwelcome side effects and drug interactions. Common side effects of fibrates include unpleasant gastrointestinal problems (including constipation and nausea). Fibrates interact with blood thinning medications (e.g. warfarin), increasing their blood thinning effect. Fibrates also interact with cholesterol-lowering medications (statins), increasing the risk of rhabdomyolysis.

Moreover, therapies utilitizing niacin and fibrate have not been very effective at raising apoA-I levels, and their ability to elevate HDL-C is variable (Meyers, C. D. and Kashyap, M. L. (2004) Curr. Opin. Cardiol. vol. 19: 366-373; Davidson, M. H. and Toth, P. P. (2004) Prog. Cardiovasc. Dis. vol. 47: 73-104). Additionally, the popularity of these drugs has been limited by the side effects associated with therapeutic dosage regimes.

A new class of drug, the cholesterol ester transfer protein (CETP) inhibitors, have been shown in preliminary studies to raise high-density lipoprotein cholesterol (HDL-C) levels (see for example: Brousseau M E, Diffenderfer M R, Millar J S, Nartsupha C, Asztalos B F, Welty F K, Wolfe M L, Rudling M, Bjorkhem I, Angelin B, Mancuso J P, Digenio A G, Rader D J, and Schaefer E J. Arterioscler Thromb Vasc Biol. 2005 May; vol. 25(5):1057-64. Epub 2005 Mar. 10). However, there are concerns that these new drugs may raise blood pressure. Torcetrapib and JTT-705, examples of the CETP inhibitors, are still in clinical trials.

Alternative methods to raise plasma apoA-I levels include administration of recombinant apoA-I Milano by intravenous infusion to patients (Nissen, S. E., et al. (2003) JAMA vol. 290: 2292-2300). The initial results suggest that apoA-I infusion can cause a significant regression of atherosclerosis. However, this therapeutic approach has serious drawbacks. Delivering drugs by intravenous infusion is clearly not practical for long-term treatment or prophylaxis. This approach will be expensive due to the high cost of producing recombinant proteins and because the drug must be administered in a clinical setting.

In another approach, negatively charged phospholipids (e.g. phosphatidylinositol (PI)) have been used to raise plasma HDL-C levels (see U.S. Pat. No. 6,828,306). Oral administration of phosphatidylinositol (PI), has been shown to effectively raise HDL-C and stimulate reverse cholesterol transport by enhancing the flux of cholesterol into HDL-C and promoting the transport of cholesterol to the liver, bile and feces (Burgess, J. W., et al. (2005) J. Lipid Res. vol. 46: 350-355; Burgess, J. W., et al. (2003) J. Lipid Res. vol. 44: 1355-1363).

However, we have found that, using conventional formulations, large oral doses of PI (e.g. about 5.6 g/day) are required to achieve clinically significant increases in plasma HDL-C (˜20%) and apoA-I (˜10%). Such large dosage amounts are somewhat impractical for long-term daily therapeutic regimes.

Therefore, there remains a need for improved pharmaceutical compositions and methods for raising plasma apoA-I levels in patients.

### SUMMARY OF THE INVENTION

The present invention provides pharmaceutical compositions and methods for raising plasma levels of apolipoprotein A-I in a mammal, e.g. for treating or preventing coronary artery disease (CAD) in mammals (especially humans).

Thus in one aspect, the present invention provides a pharmaceutical composition comprising: (a) a negatively charged phospholipid; and (b) at least one intestinal absorption enhancer; where (a) and (b) are present in such composition in amounts that render the combination effective for raising plasma levels of apolipoprotein A-I in a mammal.

In another aspect, the present invention provides a method comprising administering to a mammal: (a) a negatively charged phospholipid; and (b) at least one intestinal absorption enhancer; where (a) and (b) are administered in amounts that render the combination effective for raising plasma levels of apolipoprotein A-I in said mammal.

In another aspect, the present invention provides use of a combination comprising: (a) a negatively charged phospholipid; and (b) at least one intestinal absorption enhancer; in the preparation of a nutritional supplement or food product, where (a) and (b) are present in said nutritional supplement or food product in amounts that render the combination effective for raising plasma levels of apolipoprotein A-I in a mammal.

In another aspect, the present invention provides use for raising plasma levels of apolipoprotein A-I in a mammal of a combination of (a) a negatively charged phospholipid; and (b) at least one intestinal absorption enhancer.

In another aspect, the present invention provides use in the preparation of a medicament for raising plasma levels of apolipoprotein A-I in a mammal of a combination of (a) a negatively charged phospholipid; and (b) at least one intestinal absorption enhancer.

In another aspect, the present invention provides a commercial package comprising: (a) a negatively charged phospholipid; (b) at least one intestinal absorption enhancer; and (c) instructions for use of (a) and (b) for raising plasma levels of apolipoprotein A-I in a mammal.

### BRIEF DESCRIPTION OF THE DRAWINGS

FIG. 1: Effects of bile acids on phosphatidylinositol (PI) transport to the liver.

FIG. 2: Effects of combinations of PI and bile acids on plasma apoA-I levels.

FIG. 3: Effects of combinations of PI and selected intestinal absorption enhancers on plasma apoA-I levels.

### DETAILED DESCRIPTION

We have discovered that the efficacy of orally administered negatively charged phospholipids (PL) for raising plasma apoA-I levels can be improved by combining PL with an intestinal absorption enhancer (IAE). In some experiments, we found that administration of a combination of PL/IAE increased plasma levels of apoA-I by as much as 8-fold. Such large increases in plasma levels of apoA-I are unprecedented. These surprising results suggest that we have discovered a promising new approach to treating and preventing CAD. As discussed above, apoA-I promotes clearance of cholesterol from the body, so an increase in plasma levels of apoA-I may ameliorate atherosclerosis in a subject by reducing the size and/or number of athero-sclerotic plaques in a subject.

Thus, in accordance with the present invention, we can now achieve therapeutic endpoints (i.e. linked to increased plasma apoA-I levels) in patients using smaller dosage amounts of PL by combining PL with at least one IAE.

Herein, the expressions “raising apoA-I” and “raising HDL-C” are interchangeable.

A. Phospholipids:

Herein, the expression “negatively charged phospholipid” or “PL” refers to a phospholipid that carries a net negative charge when ionized (e.g. at physiological pH in water) and includes the corresponding non-ionized forms (e.g. salts thereof and/or protonated forms). Examples of negatively charged phospholipids therefore include but are not limited to: phosphatidylinositol (PI), phosphatidic acid (PA); phosphatidylglycerol (PG); and phosphatidylserine (PS); and salts thereof.

We have previously shown that administration of phosphatidylinositol (PI) to a mammal can raise plasma levels of HDL-C (U.S. Pat. No. 6,828,306; Burgess, J. W., et al. (2005) J. Lipid Res. vol. 46: 350-355; Burgess, J. W., et al. (2003) J. Lipid Res. vol. 44: 1355-1363). However, as noted above, large doses of PI were required to obtain clinically significant effects.

Phosphatidic acid (PA), another negatively charged phospholipid, can increase HDL-C cholesterol by about 2-fold in normocholesterolemic male Sprague-Dawley rats (Patent Abstracts of Japan, publication number 09-000206). The effective administered dose in these experiments represented 5.5% by weight of rat chow, which is equivalent to approximately 4 g/Kg body weight.

Preliminary results suggest that phosphatidylserine (PS), another negatively charged phospholipid, also raises modulates plasma HDL-C metabolism (U.S. Pat. No. 6,828,306).

Therefore, based on reports in the literature regarding the actions of several negatively charged phospholipids (PI, PA, and PS), we think that negatively charged phospholipids may constitute a class of compounds that have utility as agents for raising apoA-I.

Accordingly, the present invention can be practised using negatively charged phospholipids having the following formula I: 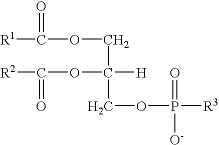
wherein
each of R¹ and R² is independently a C₁₋₂₃ hydrocarbyl group;
R³ is O⁻ or a C₁₋₁₀ hydrocarbyloxy group; and
R¹ and R² and R³ are chosen such that the phospholipid has a net charge of −1, −2 or −3;
   and pharmaceutically acceptable salts, protonated forms, and ester derivatives thereof.

Preferred phospholipids have a net charge of −1 or −2.

Suitable values for R¹ and R² include alkyl, alkenyl, and alkadienyl groups having between 3 and 23 carbon atoms, e.g. between 13 to 23 carbon atoms, or between 15 to 19 carbon atoms. For the most part, it is contemplated that R¹ and R² will be unbranched and unsubstituted. However, branching may be acceptable, provided that such branching does not interfere with the utility of the phospholipid. The presence of heteroatoms or subsituents may be acceptable, provided that such heteroatoms and substituents do not interfere with utility of the phospholipid. Suitable substituents include hydroxy, alkoxy (of an alkyl group), and mercapto (of an alkyl group). Suitable heteroatoms include O and S.

As shown in formula I, R¹ and R² are covalently bound to —C(O)— to form an acyl radical. Examples of suitable values for R¹—C(O)— and R²—C(O)— acyl radicals include but are not limited to:

tetradecanoyl [myristoyl (14:0)],

9-cis-tetradecenoyl [myristoleoyl (14:1)],

hexadecanoyl [palmitoyl (16:0)],

9-cis-hexadecenoyl [palmitoleoyl (16:1)],

9-trans-hexadecenoyl (palmitoleoyl 16:1),

octadecanoyl [stearoyl (18:0)],

6-cis-octadecenoyl [petroselinoyl (18:1)],

9-cis-octadecenoyl [oleoyl (18:1)],

9-trans-octadecadienoyl [elaidoyl(18:1)],

9-cis-12-cis-octadecadienoyl [linoleoyl (18:2)],

9-cis-12-cis-15-cis-octadecatrienoyl [linolenoyl (18:3)],

11-cis-eicosenoyl [eicosenoyl (20:1)],

5-cis-8-cis-11-cis-14-cis-eicosatetraenoyl [arachidonoyl (20:4)],

13-cis-docosenoyl [erucoyl (22:1)], and

15-cis-tetracosenoyl [nervonyl (24:1)].

Suitable values for R³ when it is a C₁₋₁₀ hydrocarbyloxy group include C₁₋₁₀ alkoxy and C₅₋₆ cycloalkyloxy. R³ may be branched or unbranched and may contain substituents or heteroatoms that do not interfere with the intended utility of the phospholipid. Suitable substituents include hydroxy, alkoxy (of an alkyl group), and mercapto (of an alkyl group). Suitable heteroatoms include O and S.

Accordingly, examples of suitable values for R³ include oxy radicals of: serine, inositol, glycerol, and sugars (such as glucose, fructose etc.).

Phospholipids of formula I can be naturally-occurring phospholipids that have been obtained from a natural source or prepared using standard chemistry. Non-naturally occurring phospholipids of formula I can also be prepared by chemical synthesis, and these phospholipids may be useful, or even preferred in some cases, for practicing the present invention.

Phospholipids of formula I can be partially hydrolyzed (i.e. to remove R¹ and/or R²) to obtain the corresponding lyso-phospholipids, which may also be useful for practicing the present invention. Therefore, in the present context, the term “phospholipid” includes both phospholipids of formula I and their corresponding lyso-phospholipids.

Phospholipids for use in the present invention may be purified or isolated or substantially pure. A compound is “substantially pure” when it is separated from the components that naturally accompany it. Typically, a compound is substantially pure when it is at least 60%, more generally 75% or over 90%, by weight, of the total material in a sample. A substantially pure phospholipid can be obtained, for example, by extraction from a natural source or by chemical synthesis. Thus, for example, a phospholipid that is chemically synthesised will generally be substantially free from its naturally associated components. Purity can be measured using any appropriate method such as column chromatography, gel electrophoresis, HPLC, etc.

However, it is not essential for a negatively charged phospholipid to be purified prior to use in the present invention, provided that the phospholipid is not associated with components that interfere substantially with its utility. The skilled person will appreciate that a natural source or partially-purified source of a negatively charged phospholipid may be used in the invention, and that the negatively charged phospholipid component may constitute a small percentage (for example 10-20%, but preferably at least 30%, 40%, 50% or more) of the total material obtained from such a source.

B: Intestinal Absorption Enhancers:

Peroral delivery of is one of the greatest challenges in biopharmaceutical research. The oral route is the preferred route of administration, but many promising drugs present low bioavailability when administered orally. As a result, intestinal absorption enhancement is an active area of research (for a recent review, see: Cano-Cebrian et al. Current Drug delivery, 2005, Volume 2, pp. 9-22).

Intestinal absorption enhancers (IAEs) are compounds that may be used to improve uptake (bioavailability) of orally administered drugs. An enormous variety of compounds have been tested as potential IAEs, including: calcium chelators; medium-chain fatty acids; medium-chain glycerides; steroidal detergents (e.g. bile acids); acylcarnitines; and chitosans and other mucoadhesive poymers (see Cano-Cebrian et al. (2005), supra).

Suitable IAEs for practising the present invention increase the efficacy of a selected negatively charged phospholipid for increasing plasma apoA-I levels by at least 10% and preferably 20%, 30%, 40%, 50%, 60%, 70%, 80%, 90%, 100%, 200%, 300%, 400% or more.

IAEs may exhibit toxicity and/or side-effects. Accordingly, they should be used in amounts and circumstances where the therapeutic benefit to the patient outweighs any undesirable side-effects.

The following classes of compounds may be useful as IAEs: bile acids; surfactants; medium chain fatty acids; and pharmaceutically acceptable salts thereof. Certain proteolytic enzymes, such as bromelain, may also be useful as IAEs (see for example: Guggi, D. and Bernkop-Schnurch, A. (2005) Int. J. Pharm. vol. 288: 141-150).

The following bile acids may be used as IAEs: deoxycholate (Uchiyama, T., et al. (1999) J. Pharm. Pharmacol. vol. 51: 1241-1250; Sakai, M., et al. (1998) J. Pharm. Pharmacol. vol. 50: 1101-1108); taurocholate (Yamamoto, A., et al. (1996) J. Pharm. Pharmacol. vol. 48: 1285-1289); chenodeoxycholate (Fricker, G., et al. (1996) Br. J. Pharmacol. vol. 117: 217-223); glycocholate (Lindhardt, K. and Bechgaard, E. (2003) Int. J. Pharm. vol. 252: 181-186; Bechgaard, E., et al. (1999) Int. J. Pharm. vol. 182: 1-5); cholate and ursodeoxycholate (Fricker, G., et al. (1996) Br. J. Pharmacol. vol. 117: 217-223); and taurodihydrofusidate (Aungst, B. J. (1993) J. Pharm Sci. vol. 82: 979-987.

Surfactants may be used as IAEs, provided that they are biocompatible. Specific examples of suitable surfactants include: Tween 20 and sodium lauryl sulfate (SLS). SLS is included in the FDA Inactive Ingredient Guide and is employed in a wide range of pharmaceutical formulations as an anionic surfactant or emulsifying agent. See: Swenson, E. S., et al. (1994) Pharm. Res. vol. 11: 1132-1142; and Anderberg, E. K., et al. (1992) J. Pharm. Sci. vol. 81: 879-887.

Suitable medium chain fatty acids (MCFAs) contain between 8 and 12 carbon atoms. Specific examples of MCFAs include: caprylate (CH₃— (CH₂)₆—COOH); caprate (CH₃— (CH₂)₈—COOH); and laurate (CH₃— (CH₂)₁₀—COOH). In many cases, the sodium salts of such MCFAs will be used, namely: sodium caprylate; sodium laurate (Yata, T., et al. (2001) J. Pharm. Sci. vol. 90: 1456-1465; Miyake, M., et al. (2003) J. Pharm. Sci. vol. 92: 911-921); and sodium caprate (see Cano-Cebrian et al. (2005) supra).

C. Therapeutic Formulations

In accordance with the present invention, a negatively charged phospholipid (PL) is combined with at least one intestinal absorption enhancer (IAE) in amounts that render the combination effective for raising plasma levels of apoA-I in a mammal.

In many cases, oral administration will be the preferred route of administration of the present combination of PL/IAE. However, alternative routes of administration (i.e. rectal and buccal administration) may be preferred in some cases. In this regard, we point out that IAEs are known to increase drug absorption via rectal administration (see for example: Matsumoto Y, et al. J. Pharmacobiodyn. 1990. Oct. 13(10):591-6) and buccal administration (for a recent review, see: Birudara; R. et al. Crit. Rev. Ther. Drug Carrier Syst. 2005. 22(3): 295-330).

Accordingly, the present combination of phospholipid/intestinal absorption enhancer (PL/IAE) may be formulated by incorporating it into a pharmaceutical composition (for oral, rectal or buccal administration), or into a supplement (such as a nutritional supplement or neutraceutical), a food product, a beverage, or the like, as known in the art. Such formulations may be used to raise plasma apoA-I levels, e.g. for preventing or treating CAD and related conditions.

A “therapeutically effective amount” refers to an amount effective, at dosages and for periods of time necessary, to achieve the desired therapeutic result, such as a clinically significant increase in plasma apoA-I levels and in turn a reduction in CAD-related disease progression. A therapeutically effective amount of the present combination of PL/IAE may vary according to factors such as the disease state, age, sex, and weight of the individual, and the ability of the particular combination of PL/IAE (i.e. the particular phospholipid and particular IAE used) to elicit a desired response in the individual. Dosage regimens may be adjusted to provide the optimum therapeutic response. A therapeutically effective amount is also one in which any toxic or detrimental effects of the compound are outweighed by the therapeutically beneficial effects.

A “prophylactically effective amount” refers to an amount effective, at dosages and for periods of time necessary, to achieve the desired prophylactic result, such as preventing or inhibiting the rate of CAD-related disease onset or progression. A prophylactically effective amount can be determined as described above for the therapeutically effective amount. For any particular subject, specific dosage regimens may be adjusted over time according to the individual need and the professional judgement of the person administering or supervising the administration of the compositions.

Thus, typically the present combination of PL/IAE is administered in an amount effective either to achieve improvement in at least one clinical sign and/or symptom of a disease caused at least in part by insufficient plasma levels of apoA-I or HDL-C (i.e. by raising plamsa levels of apoA-I and HDL-C) or to delay onset of or progression of such signs or symptoms of disease. Cure is not required, nor is it required that the improvement or delay be achievable in a single dose.

In embodiments, treatment is sufficient to increase plasma apoA-I levels by at least 20% (e.g. at least 30%, 40%, 50%, 60%, 70%, 80%, 90%, 100% or more) of the initial plamsa level of apoA-I for the patient. Plasma apoA-I levels can be measured using routine techniques.

The composition may be administered at regular intervals (e.g. daily, weekly, biweekly etc.). For example, the composition may be administered daily for a period of at least two months (e.g. at least three to six months or for at least one, two, five, ten, twenty, twenty five or more years). In some cases, such as treatment of stroke or ischemia, acute treatment may be beneficial and may require different dosages than daily or long-term treatment.

The safety and efficacy of new PL-IAE formulations can be tested using routine in vivo and in vitro techniques. For example, new PL/IAE formulations can be tested (for their ability to modulate plasma apoA-I levels) in vivo in an animal model (such as rats, pigs, mice, primates, etc.) e.g. as described herein in the Examples below, followed by tests in humans. In vitro models (such as Caco-2 cell monolayers; see e.g. Cano-Cebrian et al. (2005), supra) may also be useful for evaluating the safety and efficacy (i.e. at increasing PL absorption) of new PL/IAE formulations. In vitro studies are convenient in some respects (low cost, high throughput) and therefore may be useful in initial screening, but safety and efficacy of candidate PL/IAE formulations must be confirmed in vivo. Data obtained from in vitro assays and animal studies can be used in formulating a range of dosages for use in humans.

Typically, a unit dose may comprise, per kg of body weight of the mammal being treated: (a) between about 0.1 mg to about 140 mg of phospholipid (PL); and (b) between about 0.05 mg to about 100 mg of intestinal absorption enhancer (IAE). For example, a unit dose may comprise, per kg of body weight of the mammal being treated: (a) between about 0.1 mg to about 14 mg of PL and between about 0.1 mg to about 1.0 mg of IAE; or (b) between about 0.1 mg to about 4 mg of PL and between about 0.1 mg to about 0.5 mg of IAE. Effective doses may vary according to a number of factors (see above), and dosage regimens may be adjusted to provide the optimum therapeutic or prophylactic response.

Pharmaceutical compositions comprising the present combination of PL/IAE may include a pharmacologically acceptable excipient or carrier.

As used herein “pharmaceutically acceptable carrier” or “excipient” includes any and all solvents, dispersion media, coatings, antibacterial and antifungal agents, isotonic and absorption delaying agents, and the like that are physiologically compatible and suitable for oral administration. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Moreover, the present combination of PL/IAE can be administered in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

Specific examples of orally administrable pharmaceutical compositions include dry-filled capsules consisting of gelatin, and also soft sealed capsules consisting of gelatin and a plasticizer, such as glycerol or sorbitol. The dry-filled capsules may contain the active ingredient in the form of granules, for example in admixture with fillers, such as lactose, binders, such as starches, and/or glidants, such as talc or magnesium stearate, and optionally stabilisers. In soft capsules, the active ingredient is preferably dissolved or suspended in suitable liquids, such as fatty oils, paraffin oil or liquid polyethylene glycols, to which stabilisers may also be added.

The invention provides corresponding methods of medical treatment, in which a therapeutically effective amount of the present combination of PL/IAE is in a pharmacologically acceptable formulation for administering orally (or rectally or buccally) to a mammal subject in need thereof. In most cases, the present combination of PL/IAE will be administered simultaneously, with PL in admixture with IAE as a composition. However, this is not essential and it may be possible to administer the combination of PL/IAE as separate dosages or dosage forms. Such methods may include monitoring the subject (e.g. for plasma apoA-I levels or another sign or symptom of the condition or disorder) before, during or after treatment.

D. Other Potential Health Benefits of PL/IAE:

As discussed herein, the present combination of PL/IAE may be used to treat or prevent CAD by raising plasma levels of apoA-I and HDL-C. However, there are a number of disorders whose pathologies are caused at least in part by insufficient (or low) HDL-C levels, including: heart attack; stroke; thrombosis; dementia; metabolic syndrome; atherosclerosis; hypercholesterolemia; arteriosclerosis; dyslipidemia; and angina. Accordingly, the present combination of PL/IAE, which raises plasma levels of HDL-C, may be useful in prophylaxis or treatment of any one of the foregoing disorders. Patients having normal or even above-normal levels of apoA-I or HDL-C may still benefit from treatment to raise plasma levels of apoA-I or HDL-C, e.g. to treat or prevent any one of the foregoing disorders.

Notably, oral administration of phosphatidylserine (PS; a negatively charged phospholipid) to rats has been reported to improve age-related memory impairment (Suzuki, S., et al. (2001) J. Nutr. vol. 131: 2951-2956; Sakai, M., Yamatoya, H., and Kudo, S. (1996) J. Nutr. Sci. Vitaminol. (Tokyo) vol. 42: 47-54). In addition, orally administered PS has proven to improve memory, learning, concentration, word recall, and mood in middle-aged and elderly subjects with dementia (Heiss, W. D., et al. (1994) Dementia vol. 5: 88-98; Crook, T., et al. (1992) Psychopharmacol. Bull. vol. 28: 61-66; Amaducci, L., et al. (1991) Ann. N.Y. Acad. Sci. vol. 640: 245-249; Amaducci, L. (1988) Psychopharmacol. Bull. vol. 24: 130-134; Funfgeld, E. W., et al. (1989) Prog. Clin. Biol. Res. vol. 317: 1235-1246; Delwaide, P. J., et al. (1986) Acta Neurol. Scand. vol. 73: 136-140) or in individuals with age-related cognitive decline (Schreiber, S., et al. (2000) Isr. J. Psychiatry Relat Sci. vol. 37: 302-307; Kidd, P. M. (1999) Altern. Med. Rev. vol. 4: 144-161; Crook, T. H., et al. (1991) Neurology vol. 41: 644-649; Cenacchi, T., et al. (1993) Aging (Milano.) vol. 5: 123-133; Pepeu, G., et al. (1996) Pharmacol. Res. vol. 33: 73-8). PS improves hypothalamic-pituitary-adrenal integration, thereby improving adaptability to stress and restoring hormone rhythms which often decompensate with advancing age. Thus, in elderly men PS also partially restores thyroid-stimulating hormone and prolactin secretion rhythms (Masturzo, P., et al. (1990) Chronobiologia vol. 17: 267-274). PS can also benefit children. Two pilot studies have found that PS improved attention, behavior, learning performance, and ameliorated negative mood in 15 out of 20 children aged 4-19 years. PS also reinforced medical interventions which were already in place, such as Ritalin® therapy, and further raised the degree of functioning of these children (Kidd, P. M. (1999) Altern. Med. Rev. vol. 4: 144-161). When compared to other drugs, PS is reported to have an excellent benefit-to-risk profile and is extremely versatile in its clinical application.

However, effective intakes of PS are up to 600 mg/day when using conventional formulations of PS. Inclusion of an IAE, as disclosed herein, may decrease the effective administered dose of PS and so improve PS-based therapies.

E. Additional Active Agents:

Low plasma levels of HDL-C is only one of many risk factors associated with CAD. Other risk factors include: (1) high plasma levels of very-low-density lipoprotein cholesterol (VLDL-C) and low-density lipoprotein cholesterol (LDL-C), and (2) high plasma levels of triglycerides. Accordingly, CAD can be associated with complex dyslipidemias.

Phosphatidyl-inositol (PI) has been shown previously to be effective not only for raising plasma levels of HDL-C, as discussed above, but also for lowering plasma levels of VLDL-C and LDL-C and for stimulating lipoprotein lipase activity, i.e. for lowering plasma levels of triglycerides (see U.S. Pat. No. 6,828,306). However, in some cases, therapies that utilize more than one active agent (e.g. PL and one or more additional active agents for treating CAD) may be more suitable for treating complex dyslipidemias than therapies that utilize only one active agent. In addition, in some cases, a therapuetic regimen involving administration of two or more active agents may be more effective than administration of a single agent for achieving target plasma levels of HDL-C, VLDL-C/LDL-C, and/or triglycerides in a patient, thereby reducing the therapeutic dose required and the associated risk of undesirable side-effects. PL may act independently of or cooperatively with the additional active agent(s).

Examples of active agents that may be used together with the present PL/IAE compositions and methods include but are not limited to: 3-hydroxy-3-methyl-glutaryl coenzyme A (HMG CoA) reductase inhibitors, niacin, fibrates, and cholesterol ester transfer protein (CETP) inhibitors (e.g. torcetrapib and JTT-705). Niacin, fibrates, and cholesterol ester transfer protein (CETP) inhibitors (e.g. torcetrapib and JTT-705) are discussed above. HMG CoA reductase inhibitors are discussed below.

In the present context, the term “HMG CoA reductase inhibitor” means a competitive inhibitor of HMG CoA reductase and includes statins. “Statins” are a class of structurally related compounds that act as competitive inhibitors of HMG CoA reductase. Herein, the term “HMG CoA reductase inhibitor” includes pharmaceutically acceptable salts and ester derivatives of such compounds.

HMG CoA reductase inhibitors are known to be useful for reducing plasma levels of VLDL-C and LDL-C. These inhibitors target the enzyme HMG CoA reductase, which catalyzes an irreversible step in the cholesterol biosynthetic pathway and is therefore an important control site in cholesterol metabolism. Examples of HMG CoA reductase inhibitors suitable for use in the present invention include:
lovastatin and mevinolin (U.S. Pat. No. 4,231,938),
pravastatin sodium (U.S. Pat. No. 4,346,227),
fluvastatin (U.S. Pat. Nos. 4,739,073 and 5,354,772),
atorvastatin (U.S. Pat. No. 5,273,995),
itavastatin (European Patent No. 0304063),
mevastatin (U.S. Pat. No. 3,983,140),
rosuvastatin, velostatin, and synvinolin, and simvastatin (U.S. Pat. Nos. 4,448,784 and 4,450,171], and their pharmaceutically acceptable salts and ester derivatives. The foregoing compounds may be marketed under different names. For example, rosuvastatin is currently being marketed in the USA under the brand name “Crestor®” (AstraZeneca) and atorvastatin is currently being marketed in the USA under the brand name “Lipitor®” (Pfizer Inc.). Preferred HMG CoA reductase inhibitors for use in the present invention include rosuvastatin calcium and atorvastatin calcium.

Accordingly, PL/statin/IAE combinations and methods may be used to achieve one or more of the following effects in a subject: (1) a reduction in plasma levels of very-low-density lipoprotein (VLDL) cholesterol and low-density lipoprotein (LDL) cholesterol; (2) an increase in plasma levels of high-density lipoprotein (HDL) cholesterol levels; and (3) a reduction in plasma levels of triglycerides.

All publications and patent applications cited in this specification are herein incorporated by reference as if each individual publication or patent application were specifically and individually indicated to be incorporated by reference.

The citation of any publication is for its disclosure prior to the filing date and should not be construed as an admission that the present invention is not entitled to antedate such publication by virtue of prior invention.

### EXAMPLES

### Example I

### Use of Bile Acids to Improve Intestinal Absorption of Phosphatidylinositol

Pharmacokinetic studies were performed to determine if specific bile acids increase the absorption and transport of phosphatidylinositol (PI) to the liver. Sprague Dawley rats (300-350 g) were fasted overnight, followed by administration of 120 mg/kg ³H-PI by gavage. 50 mg/kg of the bile acids deoxycholate or glycocholate were also incorporated into the gavage. Animals were euthanized after 4 hours post-gavage at which time liver samples were harvested. PI was extracted from the liver samples using the acidic Bligh and Dyer extraction method (Bligh, E. G.; Dyer, W. J. Can. J. Biochem. vol. 37: 911-917, 1959 and Honeyman, T. W. et al., Biochem J. vol. 212: 489-498, 1983). Radioactivity present in the organic phase was measured to determine the amount of PI that was transported to the liver.

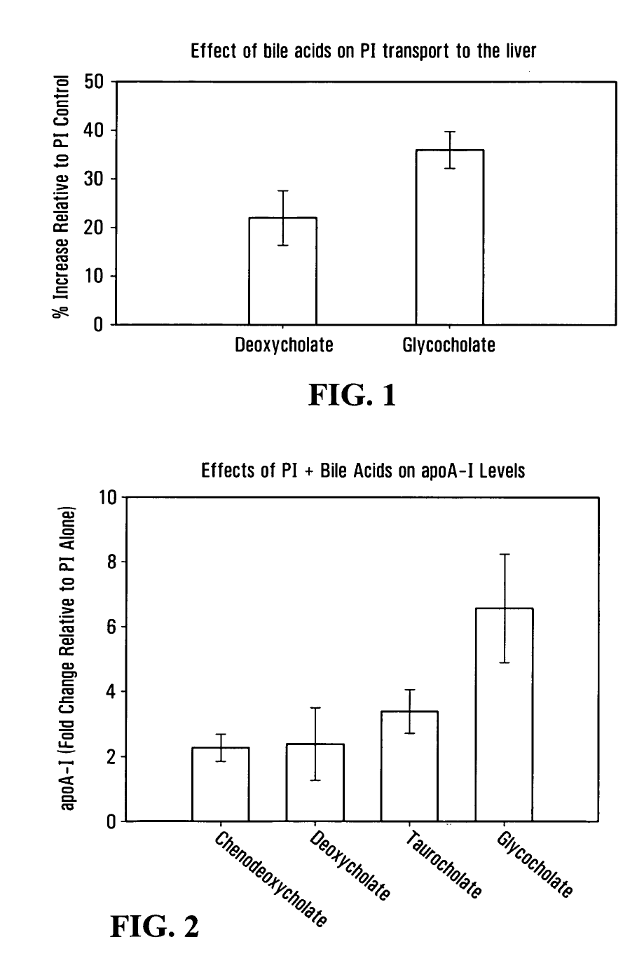

It was determined that the inclusion of bile acids significantly increased the movement of PI from the intestine to the liver (FIG. 1). Deoxycholate increased the transport of PI to the liver by 20%, whereas the use of glycocholate increased transport more substantially reaching 36% after 4 hours.

### Example II

### Use of Intestinal Absorption Inhibitors to Improve Efficacy of Phosphatidylinositol for Raising Plasma apoA-I Levels

Sprague Dawley rats (300-350 g) were fasted overnight, followed by a bolus dose of 60 mg/kg of PI incorporated into 2 g of regular maintenance chow. Individual absorption enhancers were also incorporated into the 2 g bolus dose (Table 1). The amount of bile acid added (i.e. deoxycholate, chenodeoxycholate, taurocholate and glycocholate) was chosen to maintain an equimolar amount of material (40 μmoles) per administration. Three hours after administration of PI+absorption enhancer, rats were fed 20 g of high fat chow (42% calories from fat).

TABLE 1
Absorption enhancers and amounts used to increase
the efficacy of PI

| Absorption | |
|---|---|
| Enhancer | Dose (mg/kg) |
| Deoxycholate | 50 |
| Chenodeoxycholate | 50 |
| Taurocholate | 70 |
| Glycocholate | 60 |
| SLS | 1 |
| Sodium Laurate | 15 |
| Bromelain | 120 |

All animals were maintained on this regime for two weeks, at which time animals were euthanized and blood samples collected by cardiac puncture. Blood samples were collected into EDTA coated tubes, placed on ice, followed by centrifugation at 1500×g for 15 min at 4° C. to separate plasma. Plasma samples were stored at −20° C. until analysis of apoA-I could be performed. Plasma apoA-I levels were determined by western immunoblotting.

The effect of PI and bile acids on apoA-I levels is illustrated in FIG. 2. After two weeks of treatment with PI, in combination with either chenodeoxycholate or deoxycholate, animals showed a 2-fold increase in apoA-I, relative to PI alone. This increase in efficacy is comparable to doubling the dose of PI (data not shown). More substantial apoA-I elevating effects were observed when PI was combined with either taurocholate or glycocholate, which increased apoA-I levels by 3.4 and 6.6-fold respectively.

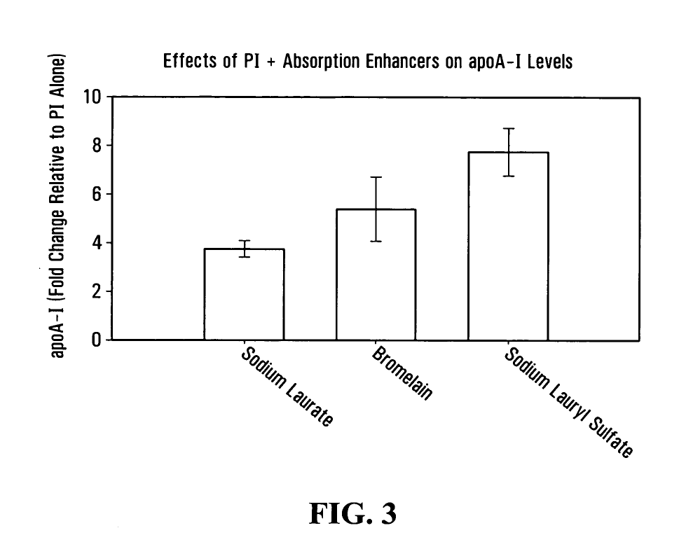

Addition of sodium laurate to PI had a comparable increase in efficacy to taurocholate and promoted a 3.7-fold increase in apoA-1 (FIG. 3). Bromelain was shown to elevate apoA-1 levels by 5.4-fold. The most dramatic increase in PI efficacy was demonstrated with the surfactant, sodium lauryl sulfate. With the inclusion of only 1 mg/kg of sodium lauryl sulfate, apoA-I levels were increased by 7.7-fold relative to animals receiving 60 mg/kg of PI alone.

### Example III

### Effects of Atorvastatin/Phosphatidylinositol Combination in HepG2 Cells

HepG2 cells respond to statins with an inhibition of HMGCOA reductase and reductions in the synthesis of cholesterol ((Bonn et al. Atherosclerosis 163.1 (2002): 59-68; Funatsu et al. Atherosclerosis 157.1 (2001): 107-15; Gerber, Ryan, and Clark Anal. Biochem. 329.1 (2004): 28-34; Maejima et al. Biochem. Biophys. Res. Commun. 324.2 (2004): 835-39; Scharnagl et al. Biochem. Pharmacol. 62.11 (2001): 1545-55; Wilcox, Barrett, and Huff J. Lipid Res. 40.6 (1999): 1078-89), increased LDL receptor mRNA (Scharnagl et al. supra), decreased apoB and (Wilcox, Barrett, and Huff supra; Funatsu et al. supra) and triglyceride secretion (Funatsu et al. supra). HepG2 cells also respond to PI (Burgess et al. J. Lipid Res. 44.7 (2003): 1355-63) with decreased production of cholesterol and cholesterol esters and with increased secretion of apoA-I.

Accordingly, candidate combinations of statins and phospholipids can be tested in HepG2 cells to assess their effects on cholesterol synthesis, LDL receptor levels, apolipoprotein secretion and LDL production.

For example, atorvastatin and phosphatidylinositol (PI) can be administered to HepG2 cells singly and in combination over a range of concentration ranges, to find concentrations of each inhibitor suitable for observing additive or synergistic effects. Typically, atorvastatin and PI are administered to HepG2 cells in amounts that, if administered singly, would typically produce less than 50% maximal response. In HepG2 cells it is reported that atorvastatin inhibits cholesterol synthesis by 45-65% when used at concentrations between 0.01 to 10 μM (Mohammadi et al. Arterioscler. Thromb. Vasc. Biol. 18.5 (1998): 783-93). Therefore, a suitable atorvastatin concentration range may include concentrations of 0.01 μM and less. PI at a concentration of 11.7 μM inhibits cholesterol synthesis by 50% in HepG2 cells (Burgess et al. supra). Therefore, a suitable PI concentration range may include 11.7 μM and less.

Briefly, HepG2 cells are seeded in 12-well-plates and grown to about 70% confluence. The monolayers of HePG2 cells are pulse-labeled with [¹⁴C]-acetate at a final concentration of 35 mM (2 mCi/L medium) and incubated for 5 h with drugs alone or in combination and at the concentrations described above. After incubation, the cells are washed three times with 1 mL of 150 mM NaCl and suspended in 1 mL of n-hexane:isopropanol (3:2 by volume). After adding 0.25 μCi of [1,2-³H]-cholesterol as an internal standard, each monolayer is extracted for 30 min. The resulting extracts are transferred to glass tubes and centrifuged at 3300 g for 20 min. The lipid phase is removed, evaporated to dryness under a stream of nitrogen, and resuspended in 1 mL of chloroform:methanol (2:1 by volume). The cell pellet is dissolved in 1 mL of 0.2 M NaOH and used for the determination of cell protein. The lipid extracts are subjected to thin-layer chromatography on silica gel plates (Merck, West Point, Pa., USA). The plates are developed with a solvent of hexane:isopropanol: formic acid (80:30:2 by volume). The spots containing triacylglycerides, nonesterified, and esterified cholesterol are visualized by iodine vapour, cut out, and counted in a scintillation counter. The data is expressed as nmol of [¹⁴C]-acetate incorporated per hour and per mg of total cell protein.

### Example IV

### Effects of Atorvastatin/Phosphatidylinositol Combination treatment in New Zealand White (NZW) rabbits

NZW rabbits respond to statins with reductions in plasma total cholesterol (Alegret et al. Eur. J. Pharmacol. 347.2-3 (1998): 283-91; Auerbach et al. Atherosclerosis 115.2 (1995): 173-80; Bocan et al. Atherosclerosis 139.1 (1998): 21-30; Verd et al. Br. J. Pharmacol. 127.6 (1999): 1479-85; Zhao and Wu Clin. Chim. Acta 360.1-2 (2005): 133-40), lipoprotein associated cholesterol (Bocan et al. supra) and triglycerides (Verd et al. supra). Statins that have been tested in rabbits include atorvastatin, fluvastatin, pravastatin and simvastatin. At doses of 10-20 mg/day in 2 kg rabbits these statins reduce plasma cholesterol levels by approximately 60% (Jorge et al. Arg Bras. Cardiol. 84.4 (2005): 314-19). Therefore, suitable doses of statins for testing for additive or synergistic effect may be on the order of less than the 10-20 mg/day dose.

The effects of orally administered PI on plasma total cholesterol, lipoprotein cholesterol and triglycerides in rabbits have not yet been properly assessed. To determine the effect of PI of plasma cholesterol in rabbits, NZW rabbits are fed diets with varying amounts of cholesterol ranging from 0.15% to 0.5% of the total daily intake, to elevate plasma cholesterol to different levels (Kolodgie et al. Arterioscler. Thromb. Vasc. Biol. 16.12 (1996): 1454-64). PI can be administered daily with chow at a dose of 120 mg/kg body weight.

To study synergistic effects of statins and PI, male NZW rabbits weighing approximately 2 kg, are divided into the following 4 groups (n=about 7): 1) hypercholes-terolemic control (no drugs administered); 2) atorvastatin; 3) PI; 4) atorvastatin and PI. The animals will be separated in individual cages and fed a standard preparation of Purina brand food, enriched with cholesterol for 4 months. Atorvastatin and PI are administered to the respective groups with chow.

Blood samples are obtained at 2 week intervals until the end of the study. The blood samples are analyzed for total cholesterol as well as lipoprotein-associated cholesterol, apolipoprotein and triglyceride levels as is standard in the literature.

### Example V

### Effects of Atorvastatin/Phosphatidylinositol Combination Treatment in Humans

The combination of statin and PI can also be tested clinically, typically orally, in humans substantially as described above (in Example IV) for rabbits. Specifically, human subjects are assigned to one of four groups: (1) control (no atorvastatin or PI to be administered); (2) atorvastatin only; (3) PI only; or (4) combination of atrovastatin/PI. Atorvastatin and PI are administered in amounts that, if administered singly, would typically produce less than 50% maximal response. Blood samples are obtained at 2 week intervals until the end of the study. The blood samples are analyzed for total cholesterol as well as lipoprotein-associated cholesterol, apolipoprotein and triglyceride levels in accordance with standard procedures in the art.

### Example VI

### Effects of Various Multi-Drug Treatments in HepG2 Cells, Rabbits and Humans

The experiments described above in Examples III to V can be used to test the effects of various combinations of negatively charged phospholipids and other active agents for treating CAD (e.g. other statins). For example, atorva-statin can be substituted with another CAD drug (e.g. another statin), and/or PI can be substituted with another negatively charged phospholipid as described herein. In order to observe synergistic effects, the subject compounds are administered in amounts that, when administered singly, would produce typically less than 50% maximal response. Note that patients may also benefit from additive effects of multi-drug therapy.

1. A pharmaceutical composition comprising:
(a) a negatively charged phospholipid; and
(b) at least one intestinal absorption enhancer;
where (a) and (b) are present in such composition in amounts that render the combination effective for raising plasma levels of apolipoprotein A-I in a mammal.

2. The pharmaceutical composition of claim 1, wherein said negatively charged phospholipid is selected from the group consisting of: phosphatidylinositol; phosphatidic acid; phosphatidylglycerol; and phosphatidylserine.

3. The pharmaceutical composition of claim 1, wherein said negatively charged phospholipid is phosphatidyl-inositol.

4. The pharmaceutical composition of claim 1, wherein said intestinal absorption enhancer is selected from the group consisting of: a bile acid or salt thereof; a surfactant or salt thereof; and a medium chain fatty acid or salt thereof.

5. The pharmaceutical composition of claim 1, wherein said intestinal absorption enhancer is sodium lauryl sulfate.

6. The pharmaceutical composition of claim 1 which comprises per unit dose: about 0.1 mg to about 140 mg of said negatively charged phospholipid; and about 0.05 mg to about 100 mg of said intestinal absorption enhancer, per kg of body weight of said mammal.

7. The pharmaceutical composition of claim 1 which comprises per unit dose: about 0.1 mg to about 14 mg of said negatively charged phospholipid; and about 0.1 mg to about 1.0 mg of said intestinal absorption enhancer, per kg of body weight of said mammal.

8. The pharmaceutical composition of claim 1 which further comprises a 3-hydroxy-3-methylglutaryl-CoA (HMG CoA) reductase inhibitor.

9. The pharmaceutical composition of claim 8, wherein the HMG CoA reductase inhibitor is the calcium salt of atorvastatin or rosuvastatin.

10. A method comprising administering to a mammal:
(a) a negatively charged phospholipid; and
(b) at least one intestinal absorption enhancer;
where (a) and (b) are administered in amounts that render the combination effective for raising plasma levels of apolipoprotein A-I in said mammal.

11. The method of claim 10, wherein said negatively charged phospholipid is selected from the group consisting of: phosphatidylinositol; phosphatidic acid;
phosphatidylglycerol; and phosphatidylserine.

12. The method of claim 10, wherein said negatively charged phospholipid is phosphatidylinositol.

13. The method of claim 10, wherein said intestinal absorption enhancer is selected from the group consisting of: a bile acid or salt thereof; a surfactant or salt thereof; and a medium chain fatty acid or salt thereof.

14. The method of claim 10, wherein said intestinal absorption enhancer is sodium lauryl sulfate.

15. The method of claim 10 which comprises per unit dose: about 0.1 mg to about 140 mg of said negatively charged phospholipid; and about 0.05 mg to about 100 mg of said intestinal absorption enhancer, per kg of body weight of said mammal.

16. The method of claim 10 which comprises per unit dose: about 0.1 mg to about 14 mg of said negatively charged phospholipid; and about 0.1 mg to about 1.0 mg of said intestinal absorption enhancer, per kg of body weight of said mammal.

17. The method of claim 10 which further comprises administering to the mammal a 3-hydroxy-3-methylglutaryl-CoA (HMG CoA) reductase inhibitor.

18. The method of claim 17, wherein the HMG CoA reductase inhibitor is the calcium salt of atorvastatin or rosuvastatin.
